(19) United States
(12) Patent Application Publication
(10) Pub. No.: US 2026/0294497 A1
(43) Pub. Date: Oct. 1, 2026

(54) SAFETY ARRANGEMENTS FOR ULTRASONICALLY-VIBRATABLE SURGICAL TOOLS

(71) Applicant: Radley Scientific Limited, Ashburton (GB)

(72) Inventors: Stephen Michael Radley YOUNG, Ashburton (GB); Michael James EDE, Newton Abbot (GB)

(21) Appl. No.: 19/478,033

(22) PCT Filed: Apr. 25, 2024
(86) PCT No.: PCT/GB2024/000017

§ 371 Date: Oct. 23, 2025

(30) Foreign Application Priority Data
Apr. 26, 2023 (GB) 2306176.5

(51) Int. Cl. A61B 17/88; A61B 17/00; A61B 17/32

(52) U.S. Cl. CPC A61B 17/8847; A61B 17/320068; A61B 2017/00075; A61B 2017/00106; A61B 2017/00119; A61B 2017/320072

(57) ABSTRACT

An ultrasonically-vibratable surgical tool for cement (7) removal in revision arthroplasty has a source of ultrasonic vibrations operatively linked to an operative tip (5) which acts on the cement (7) within a lumen (8) of a bone such as a femur (1). When acting on the cement (7), the tool vibrates substantially at a single resonant frequency. If the operative tip (5) should contact a hard material, such as a bone wall (6) of the femur (1), additional supplementary vibrations are generated at other frequencies. The tool comprises a microphone or other vibration detector to pick up these supplementary vibrations, which may be in the audible or ultrasonic ranges. When supplementary vibrations are detected at frequencies characteristic of contact between the operative tip (5) and bone, a user of the tool is alerted and operation of the tool is interrupted.

The present invention relates to improvements in ultrasonically-vibratable surgical tools for the removal of surgical cement during the revision of orthopaedic implants (revision arthroplasty). More particularly but not exclusively, it relates to arrangements to alert a user that such a tool has contacted a hard material such as bone, rather than cement, thus potentially damaging the bone.

Orthopaedic implants, such as artificial hip joints and other limb joint replacements, are frequently held in place by at least one component of the implant having an elongate, tapering shaft extending from an articulating element of the implant into a lumen of a hollow bone. For a hip joint prosthesis, one component of the implant is thus anchored into a lumen of the recipient's femur. The shaft can be secured in place by cancellous bone material ingrowing from the interior walls of the lumen. However, from 25 to 50% of implants are cemented in place within the femoral lumen, using a surgical cement based on thermoplastic poly(methyl methacrylate), often referred to as PMMA cement.

Such implants may typically be rated for a lifetime of up to 20 years. However, nowadays, the life expectancy of the recipient following surgery can easily be longer than 20 years. As life expectancies increase, so does the probability of the implant failing in service, necessitating its revision. Such “failure” rarely comprises fatigue and cracking of the metal of the implant itself. Much more often, it comprises localised failure of the cement holding the implant in place, such that the implant comes loose.

Also, the recipient of a hip joint prosthesis is likely to reach an age at which osteoporosis is beginning to present, leading to bone weakening or even bone loss. This again leads to the implant coming loose from the bone around it.

Additionally, infection of the implant-bone interface can occur, possibly due to compromised sterility during implant fixing or due to migration of bacteria from some other infection site to the trauma zone of the implant, and specifically to the bone-cement interface. This particular condition necessitates the total removal of all cement as part of the revision.

During revision, the implant or its fragments are first extracted from the bone cavity. Next, some or all of the PMMA cement remaining within the cavity must be removed. A new implant is then fitted using fresh cement, all in the same procedure. When infection is present, all cement must be removed and a temporary spacer is fitted for typically 6 weeks, while the infection is eliminated. Only then can the spacer be removed and a new implant fitted, in a two step procedure.

The old cement will often have been compacted solidly into any space between the implant and the adjacent wall of the bone cavity. Additionally, when the original implantation procedure has been carried out well, the PMMA cement will have adhered securely to the internal walls of the bone cavity. This lining of cement is awkward to remove, especially towards the distal end of a deep cavity. Sometimes part of the old cement can be left in situ as long as there is no infection and the old cement is well anchored, allowing a “cement-in-cement” fixing. Nevertheless, old cement may be more susceptible than fresh cement to forming localised defects from which failures may propagate, or may lead to inferior adhesion, either immediately or over time. Typically, PMMA cement becomes stronger over the first four or five years and then slowly weakens and degrades as it approaches 20 years old—hence the nominal 20 year rated lifetime for such implants.

The surgeon must therefore have the capability of conveniently removing old cement wherever and whenever it appears necessary, depending on its location and condition.

Only when all of the necessary PMMA cement has been removed should the implantation of a new prosthesis, using fresh cement, be permitted.

The traditional surgical approach to the removal of residual aged and hardened PMMA cement was literally to chisel the cement out of the lumen of the bone as small fragments, driving the chisel with a hammer. This is a slow, inefficient and tiring procedure for the surgeon, typically taking hours to complete.

Removal of cement with high-energy drills and burrs has also been employed, but the tips of these tools can be hard to control in the restricted space within the lumen. Either way, there is a risk of the chisel or drill bit contacting and damaging bone located adjacent the cement that is being removed. The risk and the resulting adverse effects both increase with the patient's age, especially when osteoporosis or other causes of more brittle bones are present. Additionally, prolonged surgical procedures under general anaesthesia represent a significant risk for the patient, particularly when other health issues are present.

For patients with advanced osteoporosis, and consequently with thin walls to their femurs, a surgeon may be compelled to perform an osteotomy. In this, a “controlled” aperture is cut laterally through the femur wall to enable direct access to PMMA located distal to the implant, for its removal. Repair of such an osteotomy is time consuming, adding stress and cost to the procedure and extending trauma for the patient.

A significant step forward came when the use of ultrasound in this area was developed. The friction generated between a titanium probe vibrating at over 20 kHz and cold, hard PMMA cement in contact with the probe is sufficient to heat and soften the PMMA cement into a soft, putty-like consistency, within about a second.

PMMA is a poor thermal conductor, so this heating effect remains relatively localised at the site contacted by the probe, rather than spreading out to adjacent tissues. The PMMA cement has a relatively low melting point/glass transition temperature of around 85° C., so relatively little energy has to be transferred into the cement to cause this softening.

The softened cement thus produced can readily be “scooped” out of the bone cavity, rather than requiring high-impulse hammering or high-speed drilling.

A suitably-designed ultrasonically-vibrated system can be impedance-matched specifically to the cement, leading to efficient energy deposition into the cement, but significantly less efficient transfer into adjacent bone. This further lowers the risk of damage to the bone.

Initial examples of such ultrasonically-vibratable tools were disclosed in European Patent No EP0599950.

Revision using ultrasound has been widely adopted since its introduction in the 1990s. However, more recently, it has been discovered that there may be advantages in applying different vibrational modes for this purpose.

Earlier systems used longitudinal-mode ultrasonic vibrations, which are easier to generate than other modes. Unfortunately, transfer of longitudinal-mode vibrational energy into PMMA cement seems not always to be as efficient as hoped. Additionally, there appears to be a tendency to project a proportion of the vibrational energy in the form of a “beam”, extending distally out from the tip of the tool into adjacent tissues. It was thus still possible to cause local damage to bone, if the tool was activated when its operative distal tip was aligned towards or in particular touching the bone.

Advantages have subsequently been established for tools activated by torsional-mode ultrasonic vibrations, as well as for tools vibrated in a combination of torsional and longitudinal modes.

There are indications that such tools may be superior for cement removal. It is hypothesised that this might at least in part be due to more efficient transfer of the vibrational energy into the cement. Studies into the detailed mechanisms of these processes are ongoing.

Nevertheless, with any of these tools there is still a risk of damage to the structural bone material of the surrounding femur or other hollow bone, especially if the operative distal tip of the tool is activated while touching the walls of the bone cavity. Even though the resonant ultrasonic vibrations set up in the shaft and operative distal tip of the surgical tool are substantially reflected by hard contact with the bone surface, there is still potentially some energy transfer from the tool into the bone. The operative tip of the tool could thus locally fragment or cut into the bone, especially if the bone is already weakened. Also, the energy projected into the bone could produce general weakening within a more substantial volume of bone adjacent the tool.

There is hence a need for the user of the surgical tool to be made aware that its operative tip has contacted bone or other solid material. Some surgeons have been able to take advantage of audible tones that may be created when such contact has occurred. However, this is not sufficiently reliable and is not suitable for all users, as is explained in more detail in the description below. A more reliable and widely applicable solution is therefore required.

It is hence an object of the present invention to provide apparatus for operating an ultrasonically-vibratable surgical tool, in which the apparatus detects such unplanned contacts between the activated tool and adjacent bone (or similar materials) and alerts a user of the tool accordingly. It is also an object of the present invention to provide such apparatus in which such detection results in a change in the operation of the apparatus and the associated surgical tool. A further object of the present invention is to provide a method of operation of such apparatus.

According to a first aspect of the present invention, there is provided an ultrasonically-vibratable surgical tool adapted for cement removal in revision arthroplasty, comprising
a source of ultrasonic vibrations, said source of ultrasonic vibrations being operatively connected to elongate shaft means having an operative tip located adjacent a distal end thereof, and
said elongate shaft means being adapted to transmit said ultrasonic vibrations to said operative tip,
wherein the tool comprises vibration detector means adapted to detect supplementary vibrations, said supplementary vibrations being generated as a result of contact between the operative tip and solid bone, metal or other hard material, and
wherein said vibration detector means is operatively linked to signalling means adapted to indicate to a user of the tool that said contact has occurred.

Preferably, the vibration detector means is adapted to detect supplementary vibrations having a frequency below the ultrasound range.

Preferably, the vibration detector means is adapted to detect supplementary vibrations having a frequency of below 20 kHz.

The vibration detector means may be adapted to detect supplementary vibrations having a frequency of below 15 kHz.

Optionally, the vibration detector means is adapted to detect supplementary vibrations having a frequency of at least 10 kHz.

Advantageously, the vibration detector means is adapted to detect supplementary vibrations having a frequency of around one-third of the frequency ultrasonic vibrations generated by the source and transmitted to the operative tip.

The vibration detector means may be adapted to detect supplementary vibrations in the region of 12 kHz.

Preferably, the vibration detector means is adapted to detect supplementary vibrations having a frequency of above 20 kHz.

Advantageously, the vibration detector means is adapted to detect supplementary vibrations having a frequency higher than the frequency of the ultrasonic vibrations that have been generated by the source of ultrasonic vibrations and transmitted to the operative tip.

The vibration detector means may be adapted to detect supplementary vibrations having a frequency above 36 kHz.

Preferably, the vibration detector means is adapted to detect supplementary vibrations having multiple frequencies.

The vibration detector means may then activate the signalling means in response to simultaneous detection of supplementary vibrations at two or more distinct frequencies.

The vibration detector means may be tuneable to a preselected frequency or to more than one preselected frequency.

In a preferred embodiment, the vibration detector means is adapted to detect any supplementary vibrations within a specific frequency range.

Preferably, the signalling means is then activated only when supplementary vibrations are detected within one or more preselected frequency ranges within said specific frequency range.

Preferably, the signalling means is activated only when the vibration detector means detects supplementary vibrations having an amplitude in excess of a threshold value.

Advantageously, said threshold value is selectable.

Preferably, the signalling means is only activated when the vibration detector means detects supplementary vibrations lasting for more than a pre-set duration.

Advantageously, the signalling means is only activated when the vibration detector means detects a particular supplementary vibration lasting for more than a pre-set duration.

Preferably, the vibration detector means comprises or is operatively connected to data processing means.

Advantageously, said data processing means is programmed to convert a waveform detected by the vibration detector means to a frequency spectrum.

Said data processing means may be programmed to carry out a Fast Fourier Transform on said waveform detected by the vibration detector means.

Said data processing means may be programmed to divide up said frequency spectrum into a plurality of preselected frequency ranges and optionally to register those signals falling within each of said plurality of preselected frequency ranges.

The data processing means may be programmed to register only those signals having greater than a threshold amplitude.

The data processing means may be programmed to activate the signalling means in accordance with preselected criteria.

In a preferred embodiment, the signalling means comprises audible, visible and/or haptic signalling means.

The vibration detector means may be so operatively linked to the source of ultrasonic vibrations that detection of supplementary vibrations by the vibration detector means interrupts or turns off generation of ultrasonic vibrations by said source of ultrasonic vibrations.

Advantageously, detection of supplementary vibrations by the vibration detector means interrupts generation of ultrasonic vibrations by the source of ultrasonic vibrations until said supplementary vibrations cease.

Alternatively, detection of supplementary vibrations by the vibration detector means turns off generation of ultrasonic vibrations by the source of ultrasonic vibrations until a user takes action to restart said generation.

According to a second aspect of the present invention, there is provided a method for detecting contact between an ultrasonically-vibratable surgical tool and bone or other hard material, comprising the steps of providing a surgical tool as described in the first aspect above,
detecting supplementary vibrations generated as a result of contact between an operative tip of the tool and solid bone, metal or other hard material, and
in response to detection of said supplementary vibrations, operating signalling means to indicate to a user of the tool that said contact has occurred.

In a preferred embodiment, generation of ultrasonic vibrations is interrupted or turned off in response to said detection of the supplementary vibrations.

Embodiments of the present invention will now be more particularly described by way of example and with reference to the Figures of the accompanying drawings, in which:

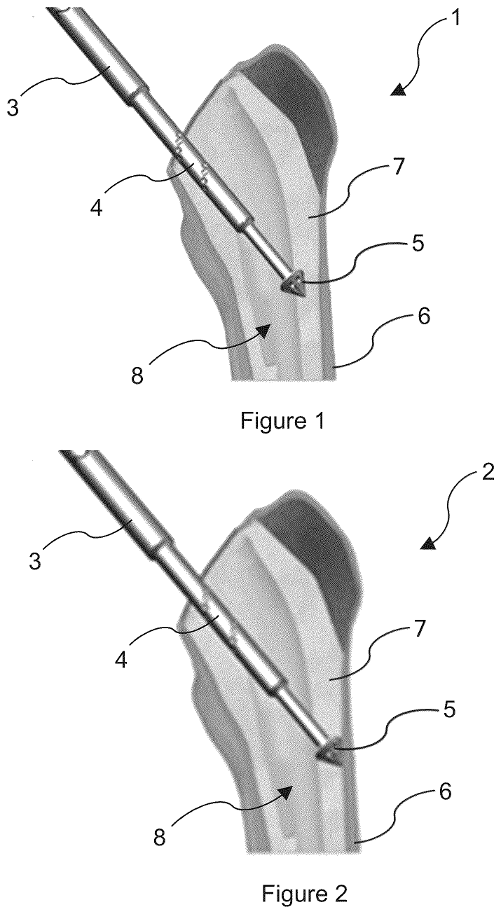

FIG. 1 is a cross-sectional elevation of an upper portion of a femur, with an ultrasonic surgical tool in use to remove remaining PMMA cement from a lumen of the femur;

FIG. 2 is a cross-sectional elevation of the upper portion of the femur of FIG. 1, with an ultrasonic surgical tool penetrating the remaining PMMA cement within the lumen of the femur so as to contact bone;

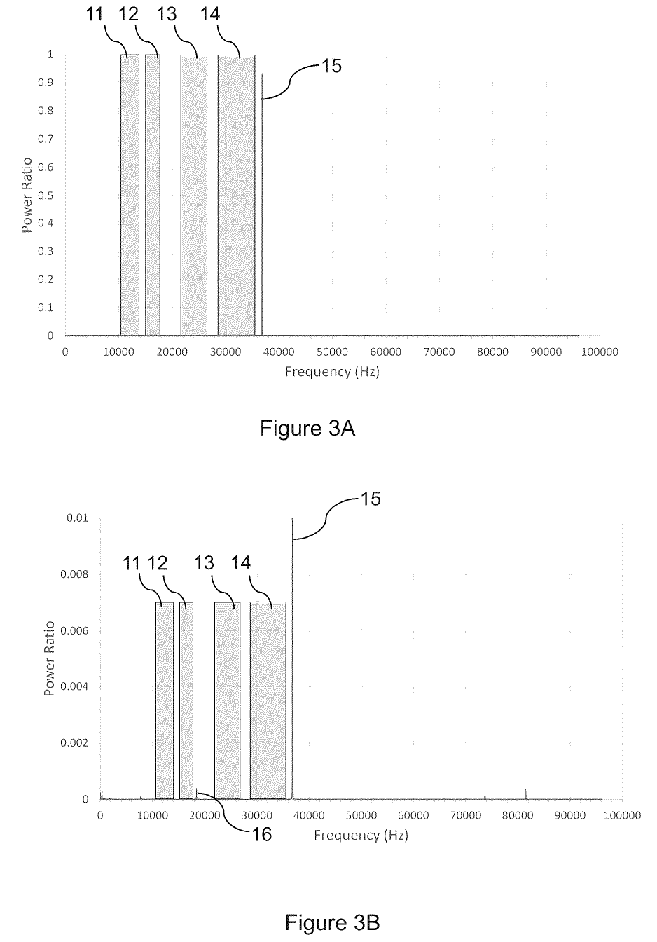

FIG. 3A is a graph of detected signal vibration frequency against the power ratio of the detected signal, normalised against the drive signal in the tool, for the tool operating normally in bulk cement;

FIG. 3B shows a portion of the graph of FIG. 3A rescaled with the power ratio axis limited to a maximum of 0.01;

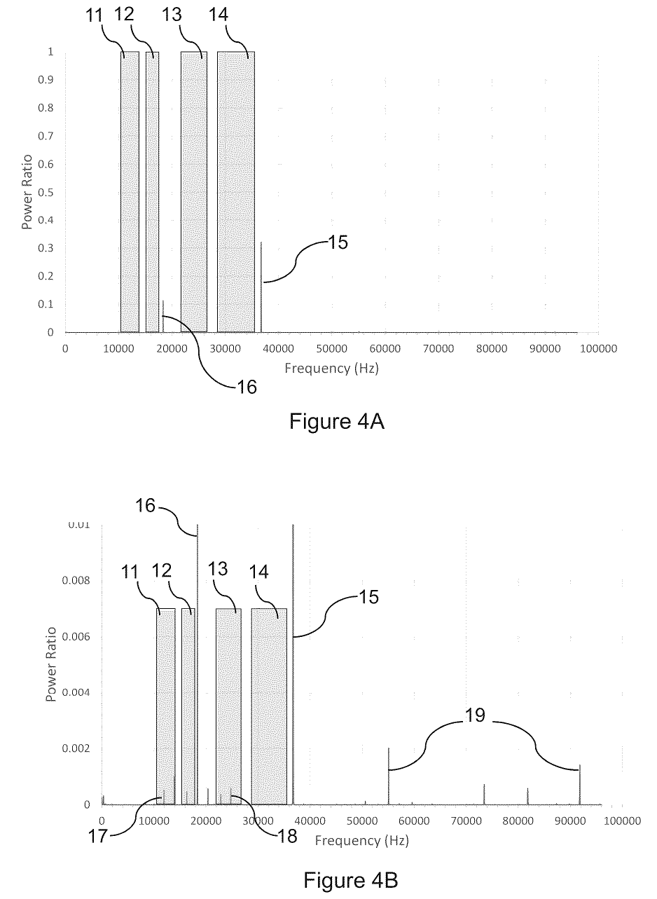

FIG. 4A is a graph of detected signal vibration frequency against the power ratio of the detected signal, as in FIG. 3A, for when the tool is first touching bone;

FIG. 4B shows a portion of the graph of FIG. 4A rescaled with the power ratio axis limited to a maximum of 0.01;

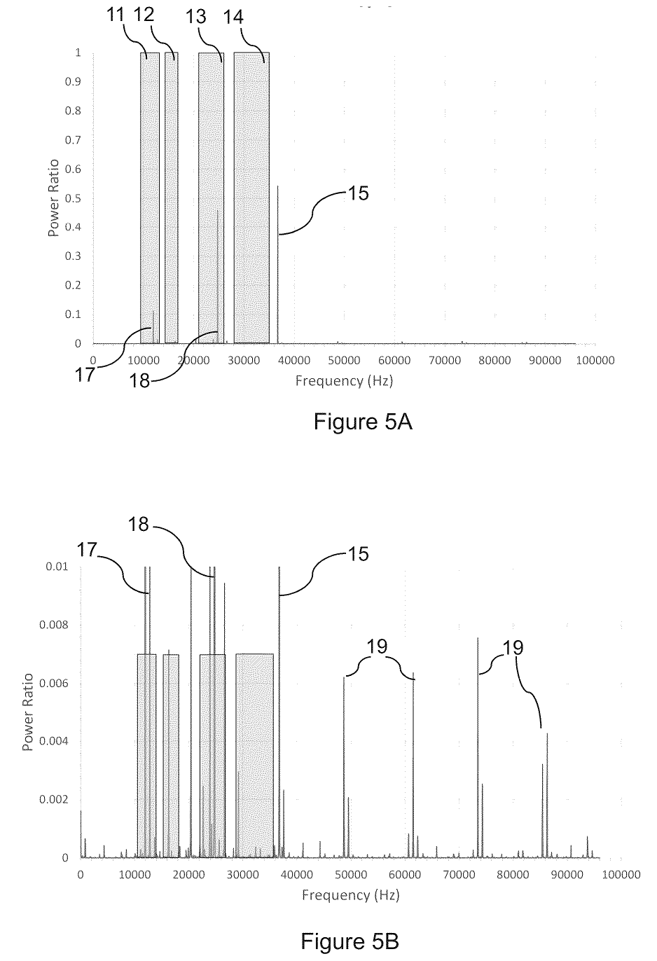

FIG. 5A is a graph of detected signal vibration frequency against the power ratio of the detected signal, as in FIG. 3A, for when the tool is fully in contact with the bone; and

FIG. 5B shows a portion of the graph of FIG. 5A rescaled with the power ratio axis limited to a maximum of 0.01.

Referring now to the Figures, and to FIG. 1 in particular, a femur 1 has had a shaft of a hip joint prosthesis extracted from a lumen 8 of the femur 1, leaving a thick layer of PMMA cement 7 coating an interior surface of a bone wall 6 of the femur 1. This cement layer 7 may need to be removed completely or in part before a replacement prosthesis is implanted into the femur 1. The implantation will use fresh cement to bond securely to both the replacement prosthesis and the bone wall 6 of the femur 1.

Part of an ultrasonically-vibratable surgical tool is also shown. This comprises an elongate cylindrical titanium shaft 3, which has a handle (not visible in FIG. 1) adjacent its proximal end for manipulation of the tool by a user. The shaft 3 is operatively connected, also adjacent its proximal end, to an ultrasound generator (also not visible in FIG. 1). An intermediate portion of the elongate shaft 3 comprises a conversion element 4, which has several helically-extending rows or chains of recesses or dimples formed into its outer surface. These have the effect of partially converting longitudinal-mode ultrasonic vibrations, which are produced by the generator and transmitted to the elongate shaft 3, into torsional-mode ultrasonic vibrations.

An operative head 5 of the tool is located at a distal end of the elongate shaft 3, distal to the conversion element 4. The operative head 5 is thus activated by a composite torsional-longitudinal mode ultrasonic vibration.

When the operative head 5 contacts the cement 7, the vibrations transmitted into the cement 7 cause rapid local heating and softening of the cement 7. This allows the substantially conically-shaped operative head 5 to be driven into the bulk of the cement 7, physically breaking up the cement 7 for subsequent removal.

Full details of this form of tool and its operation are disclosed in UK Patent Application No GB2601592 and corresponding applications in other jurisdictions. Further ultrasonically-vibratable tools, which may be used subsequently to complete the removal of the cement, are also disclosed in said application.

The ultrasound generator is programmed to vary the frequency of the ultrasonic vibrations that it provides, so as to seek out a resonant frequency within the tool. Hitting a resonant frequency of the tool maximises the amplitude of the vibrations at the tip of the operative head 5, and hence maximises the vibrational energy present in the operative head 5 for transmission therefrom into the cement 7.

The tools in question are routinely operated at a frequency in the region of 36 kHz. For context, the upper limit of human hearing is conventionally regarded as being around 20 kHz, which is therefore conventionally used as a defined lower bound for vibrations to be regarded as “ultrasonic” vibrations. However, hearing damage and simply aging can significantly reduce this upper limit of human hearing. For example, the 20 kHz limit generally applies for most people up to their late teens, but the upper frequency limit tends to fall thereafter, down to 18 kHz, 15 kHz and can even be as low as 12 kHz for mature people with no other hearing problems. The sensitivity of human hearing also tends to drop off when approaching both upper and lower frequency limits. For context, speech is generally in the range of 200 Hz to 5 kHz, and music is generally in the range of 50 Hz to 10 kHz.

FIG. 2 shows the femur in an alternative condition 2, in which the operative head 5 of the tool has been passed deeper into the layer of cement 7 than it is in FIG. 1. The tip of the operative head 5 is now in contact with the solid bone 6 of the wall of the femur 2. It is likely that vibrational energy will be transmitted into the bone 6—in effect the operative tip 5 will cut into or otherwise damage and weaken the bone 6. Damage to the bone 6 could extend as far as perforation of the bone, which would necessitate complex repair procedures, extending the duration, cost and stress of the procedure, and increase the trauma to the patient.

In practice, the operative head 5 is likely to bounce or ricochet off the bone 6 on contact, but even a short period in contact can be sufficient to cause damage to the bone 6.

As noted above, the ultrasonic vibrations set up within the tool are tuned to hit a suitable resonant frequency of the tool. This resonance frequency will be practically unaffected by contact between the operative head 5 and (rapidly-softening) cement 7. However, while the operative head 5 is in contact with hard, solid material such as bone 6, there will be a vibrational coupling effect between the tool and the bone 6, leading to a variety of additional resonances of the composite system thus (briefly) created, which will have different frequencies to the resonance originally set up within the tool alone. A common additional resonance in practice appears to be an undertone of the original resonance, having about half the frequency (thus, for the present tools this additional resonance would be at about 18 kHz).

18 kHz will be above the upper limit of hearing for almost all mature surgeons. These surgeons are hence unable to tell from generation of the undertone when they have hit bone. Even if they were to notice increased physical resistance to motion of the operative head 5, once it contacts bone, or they were to notice the operative head 5 “bouncing” off the bone 6, this is likely to be too late, after the bone 6 has already suffered damage.

Some surgeons have come to recognise hearing other, lower frequency “squeals” or “screeches” as an indication that they have touched bone 6 with the tool. However, not all surgeons appear able to detect these sounds.

In any case, the intensity of the additional resonances may be low or very low, depending for example on the exact stiffness and hardness of the cement and the bone, and the strength of the bond between them. Thus, the additional resonances may be in a theoretically audible range but too quiet to hear—those surgeons accustomed to listen out for these “squeals” often do not find them sufficiently reliable, even towards the middle of the audible range.

It has also been found that overtones and other high-frequency additional resonances may occur as a result of tool-bone contact, which are well above the audible range for any user. Here, the resonances would be inaudible regardless of their intensities.

Therefore, in the present invention, the tool is provided with a vibration detector arrangement. In an initial example this vibration detector is tuned to pick up vibrations in the region of 18 kHz. Conveniently, the vibration detector comprises an acoustic/ultrasonic microphone, mounted inside the ultrasound generator itself. Typically, due to the power cable that connects the ultrasound generator and the tool itself, the generator will be no more than three metres from the tool, making it straightforward to detect the vibrations in question.

This vibration detector arrangement is thus set to follow the amplitude of vibrations from the tool at around 18 kHz. When this amplitude exceeds a pre-set threshold, this is treated as an indication that the operative head 5 has touched bone 6 or other solid material, and the detector arrangement is therefore triggered, emitting an appropriate warning signal.

These detectors can be made far more sensitive than even the best human hearing.

This warning signal can be used to activate an audible alarm, operating at a lower frequency that is straightforward for all users to hear. Optionally, the audible alarm may have a characteristic tone or sound, rather than the substantially pure single-frequency tones of the ultrasonic vibrations, their overtones and undertones. Alternative alarm tones may be provided, selectable by the user.

The warning signal may also be used to activate a visible alarm, should the user find that more useful. Additionally or alternatively, a haptic alarm may be activated, such as a macroscopically-vibratable element in a grip of the tool, transmitting a tactile alert to the user's hand.

In some examples of the invention, a user may select which forms of alarm are to be activated.

In each case, the surgeon using the tool is rapidly and unmistakeably alerted that the operative head 5 has touched a solid material such as bone 6. The surgeon can then react by cutting off the supply of ultrasonic vibrations to the tool. Although the tool is made of titanium, it is actually not particularly sharp on a macroscopic level, and its shape can do little damage to bone when not ultrasonically activated.

The ultrasonic vibrations sent to the tool are usually turned on by the surgeon depressing a finger-operated switch on the handpiece, or alternatively depressing a foot pedal switch. It is hence generally easier and simpler to release the switch or pedal and cut off the ultrasonic vibrations, rather than to react to an alarm by retracting the operative head 5 from contact with the bone 6, possibly contacting something else undesirable in the process.

Some surgeons will wish to be in complete personal control of the ultrasonic vibrations in this way, but others may prefer a more automatic system. For an automated system, as well as (or instead of) setting off an alarm, the detector arrangement when triggered will automatically cut off the ultrasonic vibrations provided to the tool.

In one version, the ultrasound generator will then need input from the user for it to re-start—for example by tapping the foot pedal. In other versions, the ultrasonic vibrations will be shut off for a short fixed period, maybe a second or two, sufficient to allow the surgeon time to retract the (now inert) tool controllably out of contact with the bone 6 before the vibrations are automatically re-started and operation is resumed.

Although the present invention began focussed on the “undertone” vibrations at around 18 kHz, which are audible to very few users, it has been found that vibrations at other frequencies are set up as a result of contacts between the tool and bone or other hard surfaces. Some of these are at frequencies considered to be ultrasonic, including frequencies above the drive frequency (which was 36 kHz in this series of tests).

These frequencies would never have been picked up by users during existing use of these tools, even with the most acute human hearing, but a similar arrangement to that described above could be used, given the correct form of microphone, optimised to detect the relevant frequency range. Such microphones are readily available, for example being specially produced for use in bat detectors, while the standard microphone used in a smartphone appears to be capable of picking up frequencies well above the audible range.

Further examples would use more than one diagnostic frequency. The system may thus be set up to monitor multiple frequencies and to react to a significant signal at any of the possible frequencies, or alternatively to react only if there is a signal at more than one diagnostic frequency, particularly if these signals are relatively weak.

As the invention has been further developed, it has become clear that such a system needs to show a minimal rate of false positives to avoid being too irritating and inconvenient to the user, at the same time as minimising false negatives to avoid damaging tool-bone contacts.

One unexpected finding in this regard has been that the undertone at around half the drive frequency—in the example above at about 18 kHz—can be generated without there being tool-bone contact, including when the tool is operating normally on bulk cement 7. Thus, there is a risk of excessive false positives if the system is directed at the 18 kHz signal.

Further examples of the invention have therefore been developed to obviate this issue.

These have the same general layout as the initial example described above, but they track different frequencies. The operation of these versions of the invention will now be described with reference to FIGS. 3A to 5B.

The sound intensity picked up by the vibration detector during operation of the tool is processed electronically by means of the well-known Fast Fourier Transform (FFT) algorithm, which takes the sound waveform in the time domain and converts it to data in the frequency domain. This yields a frequency spectrum of the sound over a frequency range between 1 kHz and 100 kHz. The FFT can be run sixty times per second, enabling the whole of this frequency spectrum to be followed in real time. The vertical axis of the plot produced is, as shown in FIG. 3A onwards, the power ratio of the intensity of the signal detected at a particular frequency, which is normalised against the intensity of the drive signal to give a ratio between 0 and 1.0.

The detected frequencies can be subdivided into a series of “frequency windows”. Selected frequency windows to be monitored are defined so avoid the drive signal/main resonance of the tool at around 36 kHz and the undertone at around 18 kHz that has been found to be an unreliable indicator of tool-bone contact. The precise limits of these windows are currently believed to depend on the exact tool form, and it may be necessary to establish a different set of windows for different tools. FIG. 3A onwards show an example of these for the particular tool illustrated in FIGS. 1 and 2.

FIG. 3A shows the frequency spectrum for this tool operating in bulk cement. Four frequency windows 11, 12, 13, 14 to be monitored are defined, in this particular example extending from 9600 to 13200 Hz, from 14200 to 16700 Hz, from 21000 to 26000 Hz, and from 28000 to 35000 Hz respectively. (Note; these cover frequency ranges both in the “audible” range and in the low ultrasound range). In practice, each frequency window is divided into a series of “bins” covering adjacent subdivisions of the frequency range of the window, each of which is monitored individually. The “bins” are sized according to the size of the FFT, and their size and number will be a balance between accuracy and the processing power and speed of the processing unit performing the FFTs.

In bulk cement, the only detected signal 15 visible in FIG. 3A is the resonant frequency of the tool at slightly over 36 kHz. This has a power ratio of 0.94, indicating that the overwhelming majority of the input drive signal is being emitted from the tool at the preferred frequency.

FIG. 3B shows the same plot as FIG. 3B, but with the y-axis only covering power ratios between zero and 0.01. The signal 15 therefore extends way off-scale. This view also shows that in this example, the frequency windows 11, 12, 13, 14 are set up with an upper bound of power ratio=0.007. Thus, the detected signal has to exceed an amplitude of 0.07 in order to be treated as significant. (Again, the exact useful value of this upper bound may differ for different tools to that illustrated).

In FIG. 3B, there are no detectable signals within any of the frequency windows 11, 12, 13, 14. It is just possible to see a signal 16 between two of the windows 12, 13, which represents the half-frequency undertone at about 18 kHz. As noted above, this appears to be generated spontaneously at various intensities under all operating conditions, and so is not the best marker of bone contact. There are a few other very low amplitude signals, but none are anywhere near the upper bound defined for the monitored frequency windows 11, 12, 13, 14.

FIG. 4A shows a typical frequency spectrum for when the tool has just touched bone. The signal 15, representing the drive frequency/main resonance of the tool at about 36 kHz, has dropped in intensity, to a power ratio of 0.32. The signal 16 representing the half-frequency undertone at about 18 kHz has grown greatly, to a power ratio of 0.11. Both of these signals 15, 16 have actually changed frequency very slightly, but they both remain outside the monitored frequency windows 11, 12, 13, 14.

FIG. 4B shows the same plot as FIG. 4A, again with the y-axis only covering power ratios between zero and 0.01. Signals 15, 16 extend off-scale as a result. However, signals have appeared at new frequencies, such as the signals labelled 17 and 18, which are within one of the frequency windows 11, 12, 13, 14. Here, signal 17 is at about 12 kHz and signal 18 is at about 24 kHz. These are believed to be due to the tool-bone contact, for reasons explained below. Nevertheless, these signals 17, 18 at this stage have amplitudes well below the threshold of power ratio 0.007 and so they are not regarded as sufficiently significant to confirm definite tool-bone contact.

Also visible in FIG. 4B are multiple signals 19 at frequencies above the drive frequency, the relevance of which will be explained below.

FIG. 5A shows a typical frequency spectrum for when there has been sustained bone contact, showing major changes from FIGS. 3A and 4A. The main resonance signal 15 at about 36 kHz is now at a power ratio of about 0.54, but the undertone signal 16 at about 18 kHz has disappeared—confirming its unreliability as a marker of bone contact. The signal 17 at about 12 kHz has however grown greatly, to a power ratio of about 0.1, and the signal 18 at about 24 kHz has grown even further reaching a power ratio of 0.46.

FIG. 5B shows the same plot as FIG. 5A, again with the y-axis only covering power ratios between zero and 0.01, and with the threshold value of 0.07 being indicated on each of the monitored frequency windows 11, 12, 13, 14. (The frequency windows 11, 12, 13, 14 are not labelled in FIG. 5B for clarity) The signal 17 at about 12 kHz and the signal 18 at about 24 kHz clearly exceed the threshold power ratio values for the respective frequency windows 11, 13, and there are further signals that are also within one of the windows 11, 12, 13, 14 and exceed these thresholds.

Thus, in this example, there are detectable signals in three out of four frequency windows 11, 12, 13 exceeding the threshold set for significance. This is a clear indication of bone-tool contact, and can be used to trigger the contact alert signals and/or interruptions of the ultrasonic drive signal supplied to the tool, as described for the initial tool, above.

FIG. 5B also shows how the multiple signals 19 above the drive frequency have also grown significantly once the tool is fully contacting the bone. In this example, they appear at frequencies of about 48 kHz, 60 kHz, 72 kHz and so forth, which suggest that they may be overtones of the 12 kHz or 24 kHz signals. While no frequency windows have been defined to monitor these frequencies in this particular version of the invention, it is currently believed that this would not be a problematic exercise, and further development to investigate this part of the frequency spectrum is underway as of the filing date.

As for other parts of the spectrum, it has been found that frequencies much below 10 kHz are not ideal for monitoring, because (a) their actual amplitudes are usually far below those of the ultrasonic and near-ultrasonic signals monitored in the system described above, and (b) this audible frequency range can be very “busy” even when the tool is merely operating on bulk cement.

The threshold levels for the defined frequency windows can be set by reference to the intensity of the detected drive signal as described above, or can be set as absolute values. Normalising by reference to the drive signal has the advantage that it eliminates effects due to the exact distance from the microphone to the tool, or due to the surgeon possibly standing in a direct line between the microphone and the tool. In either case, the threshold set is an empirical compromise between minimising false positives and minimising the time taken to alert the user to bone contact.

The sensitivity of the system can be adjusted by adjusting the number of consecutive “positives” (i.e. a signal exceeding the threshold, detected within a particular bin, in consecutive FFT runs) that are required before a contact alert is generated. The conditions necessary for ending an alert can also be adjusted, so that the system will produce a single continuous alert in preference to rapidly-repeated brief alerts.

In the currently preferred versions of the invention, the definition used for a “genuine, sustained” bone contact worthy of an alert is to require a contact signal in a particular bin to exceed the threshold for five successive cycles of the FFT operation. Since the FFT is currently being run at sixty cycles per second, this still provides a very rapid warning to the user. At present, these positives may be in any one, two or three of the frequency windows, or all four 11, 12, 13, 14. The approach taken is to “accumulate” positives in respect of each bin until one or more reach five positives. If there are no values above the threshold for the entire frequency range, all the bins are re-set to zero.

The number of consecutive positives required can thus be adjusted to tailor the sensitivity of the system. Optionally, this can be selected by the surgeon, for example choosing high sensitivity if the subject's bone is known to be very fragile or already damaged, or low sensitivity for procedures where they know that briefly touching bone is quite likely, or for procedures with a lower risk of bone perforation, such as scraping cement out of a bone lumen in a proximal direction, rather than piercing cement in a distal direction, as in FIGS. 1 and 2.

Setting up criteria that work with a range of tools is considered to be important, to ensure that the invention works well for each tool. It is preferable for the monitored frequency windows to cover the signals produced by the whole range of likely contacts, rather than requiring different set-ups for different tools, for example. The exact tool geometries are believed to be important, as well as all the different degrees of tool contact that may be produced. Factors affecting this interaction are believed to include:
a) Contact force
b) Contact angle
c) Bone damping by cement
d) Bone properties including density, thickness and type (note: the cancellous bone that may have grown around an implant is generally so soft that it is not detected by this approach, and only contacts with cortical bone will be registered—this is actually beneficial, as any cancellous bone that has formed will also need to be removed before a new prosthesis is implanted, as a general rule).

The above variations in the exact bone-tool interactions are believed to be behind the unreliability of the traditional approach of the surgeon listening for audible-range contact “squeals”.

The present invention thus allows undesirable contacts between an ultrasonically-activated tool and adjacent bone or other solid material to be minimised, more reliably and conveniently than existing approaches such as listening out for particular squeals or screeches, which may or may not be produced at an audible intensity.

1. An ultrasonically-vibratable surgical tool adapted for cement removal in revision arthroplasty, comprising
a source of ultrasonic vibrations, said source of ultrasonic vibrations being operatively connected to an elongate shaft having an operative tip located adjacent a distal end of the elongate shaft, and
said elongate shaft being adapted to transmit said ultrasonic vibrations to said operative tip,
wherein the tool comprises a vibration detector adapted to detect supplementary vibrations, said supplementary vibrations being generated as a result of contact between the operative tip and solid bone, metal or other hard material, and
wherein said vibration detector is operatively linked to a signalling element adapted to indicate to a user of the ultrasonically-vibratable surgical tool that said contact has occurred.

2. The ultrasonically-vibratable surgical tool as claimed in claim 1, wherein the vibration detector is adapted to detect supplementary vibrations having a frequency below the ultrasound range.

3. The ultrasonically-vibratable surgical tool as claimed in claim 1, wherein the vibration detector is adapted to detect supplementary vibrations having a frequency of below 20 kHz.

4. The ultrasonically-vibratable surgical tool as claimed in claim 3, wherein he vibration detector is adapted to detect supplementary vibrations having a frequency of below 15 kHz.

5. An The ultrasonically-vibratable surgical tool as claimed in claim 1, wherein the vibration detector is adapted to detect supplementary vibrations having a frequency of greater than 10 kHz.

6. The ultrasonically-vibratable surgical tool as claimed in claim 1, wherein the vibration detector is adapted to detect supplementary vibrations having a frequency of around one-third of the frequency ultrasonic vibrations that have been generated by the source of ultrasonic vibrations and transmitted to the operative tip.

7. (canceled)

8. The ultrasonically-vibratable surgical tool as claimed in claim 1, wherein the vibration detector is adapted to detect supplementary vibrations having a frequency of above 20 kHz.

9. The ultrasonically-vibratable surgical tool as claimed in claim 8, wherein the vibration detector is adapted to detect supplementary vibrations having a frequency higher than the frequency of the ultrasonic vibrations that have been generated by the source of ultrasonic vibrations and transmitted to the operative tip.

10. The ultrasonically-vibratable surgical tool as claimed in claim 8, wherein the vibration detector is adapted to detect supplementary vibrations having a frequency above 36 kHz.

11. The ultrasonically-vibratable surgical tool as claimed in claim 1, wherein the vibration detector is adapted to detect supplementary vibrations having multiple frequencies.

12. The ultrasonically-vibratable surgical tool as claimed in claim 11, wherein the vibration detector activates the signalling element in response to simultaneous detection of supplementary vibrations at two or more distinct frequencies.

13. The ultrasonically-vibratable surgical tool as claimed in claim 1, wherein the vibration detector is tuneable to at least one preselected frequency.

14. The ultrasonically-vibratable surgical tool as claimed in any claim 1, wherein the vibration detector is adapted to detect any supplementary vibrations within a specific frequency range.

15. The ultrasonically-vibratable surgical tool as claimed in claim 14, wherein the signalling element is activated only when supplementary vibrations are detected within one or more preselected frequency ranges within the specific frequency range.

16-17. (canceled)

18. An The ultrasonically-vibratable surgical tool as claimed in claim 1, wherein the signalling element is only activated when the vibration detector detects supplementary vibrations lasting for more than a pre-set duration.

19. (canceled)

20. The ultrasonically-vibratable surgical tool as claimed in claim 1, wherein the vibration detector comprises or is operatively connected to data processing means.

21-25. (canceled)

26. The ultrasonically-vibratable surgical tool as claimed in claim 1, wherein the signalling element comprises audible, visible and/or haptic signalling means.

27. The ultrasonically-vibratable surgical tool as claimed in claim 1, wherein the vibration detector is so operatively linked to the source of ultrasonic vibrations that detection of supplementary vibrations by the vibration detector interrupts or turns off generation of ultrasonic vibrations by said source of ultrasonic vibrations.

28. A method for detecting contact between an ultrasonically-vibratable surgical tool and solid bone, metal or other hard material, comprising the steps of providing an ultrasonically-vibratable surgical tool as claimed in claim 1,
detecting supplementary vibrations generated as a result of contact between an operative tip of the ultrasonically-vibratable tool and solid bone, metal or other hard material, and
in response to detection of said supplementary vibrations, operating the signalling element to indicate to a user of the ultrasonically-vibratable tool that said contact has occurred.

29. The method as claimed in claim 28, wherein generation of ultrasonic vibrations by the source of ultrasonic vibrations is interrupted or turned off in response to the detection of the supplementary vibrations.
